(19) United States
(12) Patent Application Publication
(10) Pub. No.: US 2026/0306817 A1
(43) Pub. Date: Oct. 8, 2026

(54) Palpable Concave Port Septum

(71) Applicant: Bard Peripheral Vascular, Inc., Franklin Lakes, NJ (US)

(72) Inventors: Ian N. Thomas, West Bountiful, UT (US); Bryon Ray Densley, Rochester, MN (US)

(21) Appl. No.: 18/996,881

(22) PCT Filed: Jul. 20, 2022
(86) PCT No.: PCT/US22/37690

§ 371 Date: Jan. 17, 2025

(51) Int. Cl. A61M 39/02

(52) U.S. Cl. CPC A61M 39/0208; A61M 2039/0205; A61M 2039/0238; A61M 2205/0216; A61M 2207/00; A61M 2210/04; A61M 2210/12

(57) ABSTRACT

Embodiments disclosed herein are directed to subcutaneous vascular access ports including a septum having a concave section to the top surface, and an annular palpation rim. The septum can define a radially symmetrical shape extending about a central transverse axis. The top-most edge of the palpation rim can define a top-most edge of the port. The rim can include an outer surface extending outwards from the top-most edge, an inner surface extending radially inward from the top-most edge of the rim. The concave surface of the septum and/or the rim can provide a palpation feature to indicate a location of the port, or more specifically a location of the reservoir disposed below. The concave septum provides a palpation feature while reducing the overall height of the port. This can be important for low-profile ports that are designed to reduce stretching of the skin surface which can lead to skin erosion.

### SUMMARY

Briefly summarized, embodiments of the present invention are directed to subcutaneous vascular access devices, or “ports,” including a septum having a concave top surface and an annular palpation rim. The septum can define a radially symmetrical shape extending about a central transverse axis. The top-most edge of the palpation rim can define a top-most edge of the port. The rim can include an outer surface extending from the top-most edge of the rim to a radially outer-most edge of the septum, and an inner surface extending from the top-most edge of the rim to the central transverse axis.

Advantageously, the concave surface of the septum can provide a palpation feature to indicate a location of the port, or more specifically a location of the reservoir disposed transversely below the septum, for percutaneous access with a needle. Further, the concave septum provides a palpation feature without unnecessarily increasing the overall transverse height of the port. This can be important for low-profile ports that are designed to reduce stretching of the skin surface which can lead to skin erosion.

Disclosed herein is a vascular access port including, a port body having a reservoir, and a septum positioned over the reservoir and captured by the port body, the septum including, a concave top surface configured for penetration by a needle to permit fluid communication between the needle and the reservoir, and a continuous rim extending around a central transverse axis of the concave surface.

In some embodiments, the septum defines a radially symmetrical shape extending about the central transverse axis.

In some embodiments, the continuous rim extends transversely outwards from an upper-most surface of the port body, extending away from the access port.

In some embodiments, the upper surface of the continuous rim defines an upper-most surface of the vascular access port.

In some embodiments, the septum including the continuous rim is formed of a compliant material selected from a group consisting of a plastic, polymer, elastomer, organic or synthetic rubber, or silicone rubber.

In some embodiments, the continuous rim includes an outer surface, the outer surface extends between an upper-most edge and a radially outer-most edge, and an inner surface, the inner surface extends between the upper-most edge and the central transverse axis.

In some embodiments, the outer surface defines a concave cross-sectional profile.

In some embodiments, the outer surface defines a convex cross-sectional profile.

In some embodiments, the outer surface defines a continuous incline relative to the central transverse axis.

In some embodiments, the inner surface defines a convex cross-sectional profile.

In some embodiments, the inner surface includes sigmoid cross-sectional profile including a convex section and a concave section.

In some embodiments, the inner surface includes one of a discontinuous change in incline or a continuous change in incline.

In some embodiments, one or both of the outer surface and the inner surface includes a vertical section extending parallel to the central transverse axis.

In some embodiments, the concave surface includes a portion that extends below an upper-most surface of the port body.

In some embodiments, the septum includes flange extending radially outward from a side surface of the septum.

In some embodiments, the port is configured to be disposed subcutaneously and further includes a catheter coupled to a port stem and in fluid communication with the reservoir, the distal tip of the catheter disposed within a vasculature.

Also disclosed is a method of manufacturing a vascular access port including, forming a port body defining a reservoir, and coupling a septum with the port body, the septum positioned over the reservoir and having a top surface having a concave portion and configured for penetration by a needle to permit fluid communication between the needle and the reservoir, and a continuous rim extending around a central transverse axis of the concave surface.

In some embodiments, the septum defines a radially symmetrical shape extending about the central transverse axis.

In some embodiments, the continuous rim extends transversely outwards from an upper-most surface of the port body, extending away from the port body.

In some embodiments, the upper surface of the continuous rim defines an upper-most surface of the vascular access port.

In some embodiments, the septum including the continuous rim is formed of a compliant material selected from a group consisting of a plastic, polymer, elastomer, organic or synthetic rubber, or silicone rubber.

In some embodiments, the continuous rim includes an outer surface extending between an upper-most edge and a radially outer-most edge, and an inner surface extend between the upper-most edge and the central transverse axis.

In some embodiments, the outer surface defines a concave cross-sectional profile.

In some embodiments, the outer surface defines a convex cross-sectional profile.

In some embodiments, the outer surface defines a continuous incline.

In some embodiments, the inner surface defines a convex cross-sectional profile.

In some embodiments, the inner surface includes sigmoid cross-sectional profile including a convex section and a concave section.

In some embodiments, the inner surface includes one of a discontinuous change in incline or a continuous change in incline.

In some embodiments, one or both of the outer surface and the inner surface includes a vertical section extending parallel to the central transverse axis.

In some embodiments, the concave surface includes a portion that extends below an upper-most surface of the port body.

In some embodiments, the septum includes flange extending radially outward from a side surface of the septum.

Also disclosed is a method of accessing a vascular access port including, palpating a septum of the vascular access port to determine a rim of the septum and a concave center point of the septum, wherein the rim defines a edge of the vascular access port, inserting an access needle into a center point of the septum, penetrating the septum, and accessing a reservoir of the vascular access port to provide fluid communication with the needle.

In some embodiments, the rim extends continuously about the transverse center point of the septum.

In some embodiments, a diameter of the top-most edge of the septum is equal to, or less than a lateral diameter of the reservoir.

In some embodiments, the concave center point of the septum extends transversely below an upper-most edge of a body of the vascular access port.

### DRAWINGS

A more particular description of the present disclosure will be rendered by reference to specific embodiments thereof that are illustrated in the appended drawings. It is appreciated that these drawings depict only typical embodiments of the invention and are therefore not to be considered limiting of its scope. Example embodiments of the invention will be described and explained with additional specificity and detail through the use of the accompanying drawings in which:

FIG. 1A shows a perspective view of a port including a palpable concave septum, in accordance with embodiments disclosed herein.

FIG. 1B shows a cross-section view of a port including a palpable concave septum, in accordance with embodiments disclosed herein.

FIG. 2A shows a perspective view of a port including a palpable concave septum, in accordance with embodiments disclosed herein.

FIG. 2B shows a cross-section view of a port including a palpable concave septum, in accordance with embodiments disclosed herein.

FIG. 3A shows a perspective view of a port including a palpable concave septum, in accordance with embodiments disclosed herein.

FIG. 3B shows a cross-section view of a port including a palpable concave septum, in accordance with embodiments disclosed herein.

FIG. 4A shows a perspective view of a port including a palpable concave septum, in accordance with embodiments disclosed herein.

FIG. 4B shows a cross-section view of a port including a palpable concave septum, in accordance with embodiments disclosed herein.

FIG. 5 shows a cross-section view of a palpable concave septum, in accordance with embodiments disclosed herein.

FIG. 6 shows a cross-section view of a palpable concave septum, in accordance with embodiments disclosed herein.

FIG. 7 shows a cross-section view of a palpable concave septum, in accordance with embodiments disclosed herein.

FIG. 8 shows a cross-section view of a palpable concave septum, in accordance with embodiments disclosed herein.

### DESCRIPTION

Before some particular embodiments are disclosed in greater detail, it should be understood that the particular embodiments disclosed herein do not limit the scope of the concepts provided herein. It should also be understood that a particular embodiment disclosed herein can have features that can be readily separated from the particular embodiment and optionally combined with or substituted for features of any of a number of other embodiments disclosed herein. It is understood that the drawings are diagrammatic and schematic representations of exemplary embodiments of the invention, and are neither limiting nor necessarily drawn to scale.

Regarding terms used herein, it should also be understood the terms are for the purpose of describing some particular embodiments, and the terms do not limit the scope of the concepts provided herein. Ordinal numbers (e.g., first, second, third, etc.) are generally used to distinguish or identify different features or steps in a group of features or steps, and do not supply a serial or numerical limitation. For example, “first,” “second,” and “third” features or steps need not necessarily appear in that order, and the particular embodiments including such features or steps need not necessarily be limited to the three features or steps. Labels such as “left,” “right,” “top,” “bottom,” “front,” “back,” and the like are used for convenience and are not intended to imply, for example, any particular fixed location, orientation, or direction. Instead, such labels are used to reflect, for example, relative location, orientation, or directions. Singular forms of “a,” “an,” and “the” include plural references unless the context clearly dictates otherwise. Also, the words “including,” “has,” and “having,” as used herein, including the claims, shall have the same meaning as the word “comprising.” In the following description, the terms “or” and “and/or” as used herein are to be interpreted as inclusive or meaning any one or any combination. As an example, “A, B or C” or “A, B and/or C” mean “any of the following, A, B, C, A and B, A and C, B and C, A, B and C.” An exception to this definition will occur only when a combination of elements, components, functions, steps or acts are in some way inherently mutually exclusive.

With respect to “proximal,” a “proximal portion” or a “proximal end portion” of, for example, a catheter disclosed herein includes a portion of the catheter intended to be near a clinician when the catheter is used on a patient. Likewise, a “proximal length” of, for example, the catheter includes a length of the catheter intended to be near the clinician when the catheter is used on the patient. A “proximal end” of, for example, the catheter includes an end of the catheter intended to be near the clinician when the catheter is used on the patient. The proximal portion, the proximal end portion, or the proximal length of the catheter can include the proximal end of the catheter; however, the proximal portion, the proximal end portion, or the proximal length of the catheter need not include the proximal end of the catheter. That is, unless context suggests otherwise, the proximal portion, the proximal end portion, or the proximal length of the catheter is not a terminal portion or terminal length of the catheter.

With respect to “distal,” a “distal portion” or a “distal end portion” of, for example, a catheter disclosed herein includes a portion of the catheter intended to be near or in a patient when the catheter is used on the patient. Likewise, a “distal length” of, for example, the catheter includes a length of the catheter intended to be near or in the patient when the catheter is used on the patient. A “distal end” of, for example, the catheter includes an end of the catheter intended to be near or in the patient when the catheter is used on the patient. The distal portion, the distal end portion, or the distal length of the catheter can include the distal end of the catheter; however, the distal portion, the distal end portion, or the distal length of the catheter need not include the distal end of the catheter. That is, unless context suggests otherwise, the distal portion, the distal end portion, or the distal length of the catheter is not a terminal portion or terminal length of the catheter.

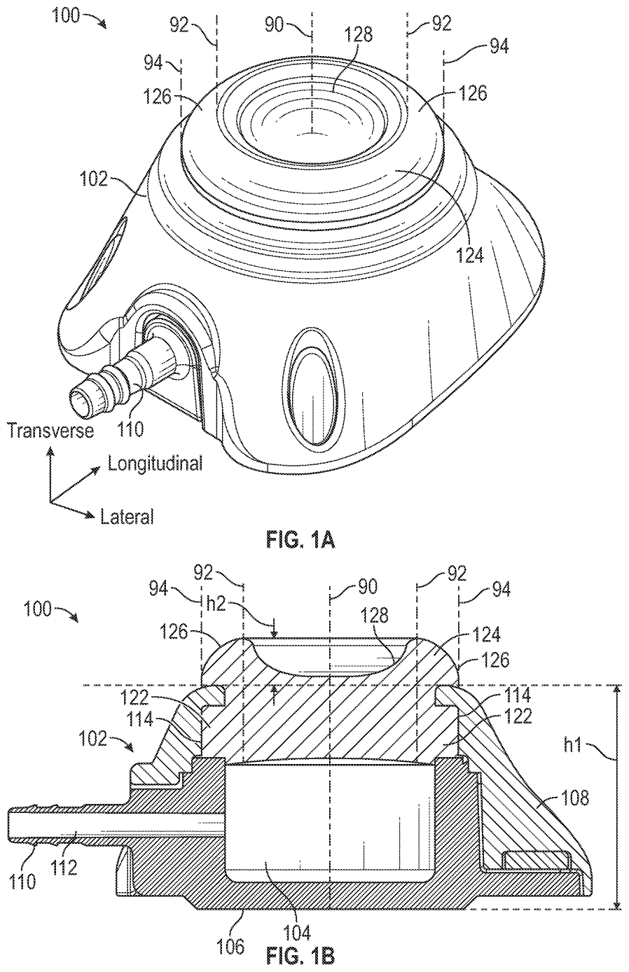

To assist in the description of embodiments described herein, as shown in FIG. 1A, a longitudinal axis extends substantially parallel to an axial length of a stem of the port. A lateral axis extends normal to the longitudinal axis, and a transverse axis extends normal to both the longitudinal and lateral axes. A vertical axis can extend parallel to a transverse axis. A horizontal axis can extend perpendicular to the vertical axis. A horizontal plane can be defined by the longitudinal and lateral axes. A vertical plane can extend perpendicular to the horizontal plane.

As used herein, a “top” or “upper” surface or direction is a surface or direction extending along a transverse axis that is relatively closer to the skin surface when the port 100 is placed subcutaneously, i.e. a surface or direction that is relatively further from the base 106 of the port 100. Similarly, a “bottom” or “lower” surface or direction is a surface or direction extending along a transverse axis that is relatively further from the skin surface when the port 100 is placed subcutaneously, i.e. a surface or direction that is relatively closer to the base 106 of the port 100.

Unless defined otherwise, all technical and scientific terms used herein have the same meaning as commonly understood by those of ordinary skill in the art.

FIGS. 1A-1B shows an embodiment of a subcutaneous vascular access device, or “port” 100 generally including a port body 102 defining a reservoir 104 and including a port stem 110. The port stem 110 can extend from the port body 102 along a longitudinal axis and can define a stem lumen 112 that is in fluid communication with the reservoir 104.

In an embodiment, the body 102 can comprise a base portion (“base”) 106 and a housing 108. The housing 108 can be formed of the same material or from a different material from that of the base 106. In an embodiment the housing 108 can be overmolded on to the base 106. In an embodiment, the housing 108 can engage the base 106 in one or more of a press-fit, snap-fit, or interference fit engagement. In an embodiment, the housing 108 can be secured to the base 106 using adhesive, bonding, welding, ultrasonic welding, or the like. In an embodiment, one of the base 106 or the housing 108 can define the reservoir 104. In an embodiment, the base 106 and the housing 108 can co-ordinate to define the reservoir 104.

In an embodiment, the body 102 can define a transverse height (h1) extending between a bottom-most surface and a top-most surface of the body 102. As noted, the port 100 can be designed to maintain as low of a transverse height (h1) profile as possible to mitigate stretching of the skin when placed subcutaneously and mitigate skin erosion.

In an embodiment, the port 100 can further include a needle-penetrable septum 120 disposed over the reservoir 104 and configured to provide percutaneous access to the reservoir 104 by an access needle (not shown). When the port 100 is placed subcutaneously, the access needle can extend transversely through a skin surface and can penetrate the septum 120 to provide fluid communication with the reservoir 104. A fluid can then pass through the access needle, into the reservoir 104, through the port stem lumen 112 and into a lumen of a catheter coupled to the port stem 110. A distal tip of the catheter can be disposed within a vasculature of a patient.

In an embodiment, the septum 120 can define a radially symmetrical shape extending about a central transverse axis 90. As shown in FIG. 1B, a cross-sectional profile of an upper surface of the septum 120 can extend between a central transverse axis 90 and a radially outer-most edge 94. In an embodiment, at least a portion, e.g. a central portion, of the top surface of the septum 120 can define a concave shape. As such, an upper-most, or top-most edge 92, of the septum 120 can be disposed radially outwards from the central transverse axis 90 to define a palpation ring 124. In an embodiment, an entire upper surface of the septum 120 can define a concave surface. As used herein, a top-most edge is an edge or surface disposed transversely outermost and away from a base 106 of the port 100.

In an embodiment, a portion of the top surface of the septum 120 disposed at the central transverse axis 90 can be transversely aligned with a top-most edge of the port body 102. As such, the palpation ring 124 can extend from the top-most surface of the port body 102 by a second height (h2). In an embodiment, the septum 120 can be formed of a relatively compliant material such as a plastic, polymer, elastomer, organic or synthetic rubber, silicone rubber, or the like.

To note, when the port 100 is placed subcutaneously, the skin surface tissues are stretched and distended over the port 100 to accommodate the transverse height of the port 100. Skin erosion can occur where the skin surface tissues contact the top most edges of the port 100 and are stretched thereover. Low-profile ports, i.e. ports with a reduced transverse height, have been developed to mitigate the stretching and skin erosion. As described herein, a top-most edge of the port 100 can be defined by the top-most edge 92 of the palpation ring 124 which is formed of the relatively compliant material of the septum 120. As such the compliant palpation ring 124 can elastically deform, dispersing the pressure across the skin surface tissues, mitigating skin erosion.

In an embodiment, the septum 120 can include a flange 122 extending from a side surface of the septum 120 and extending annularly about the central transverse axis 90. The flange 122 can engage a channel 114 defined by the port body 102 to secure the septum 120 in place over the reservoir 104. In an embodiment, the base 106 and the housing 108 can co-operate to define the channel 114.

In an embodiment, a cross-sectional profile of the palpation ring 124 can define an inner surface 128 and an outer surface 126. An inner surface 128 of the palpation ring 124 can extend from the central transverse axis 90 to a top-most edge 92. An outer surface 126 of the palpation ring 124 can extend from the top-most edge 92 to the outer-most edge 94. As previously noted, the septum 120 defines a radially symmetrical shape about the central transverse axis 90.

As shown in FIG. 1B, in an embodiment, the inner surface 128 of the palpation ring 124 can define a concave cross-sectional shape. In an embodiment, the outer surface 126 of the palpation ring 124 can define a convex cross-sectional shape. Advantageously, the convex shape of the outer surface 126 of the palpation ring 124 can further disperse the pressure across the skin surface tissues stretched over the port 100 and can mitigate skin erosion when the port is placed subcutaneously.

When the port 100 is disposed subcutaneously, the palpation ring 124 of the septum 120 can extend transversely outwards from the port 100, away from the port body 102, and can be palpated by a user to determine a location of the port 100. More specifically, the palpation ring 124 can provide a concave central portion to the top surface of the septum 120. Advantageously, the concave top surface of the septum 120 can direct the user's digit and/or access needle towards the central transverse axis 90 and align the digit or access needle with the reservoir 104 disposed transversely below the septum 120.

In an embodiment, the top-most edge 92 of the palpation ring 124 can be transversely aligned with, or aligned just inside of, a vertical wall of the reservoir 104. As such, the palpation ring 124 can indicate a horizontal footprint of the reservoir 104 disposed below. Advantageously, the concave surface of the septum 120 defined by the top-most edge 92 of the palpation ring 124 can indicate to a user the location of the reservoir 104 and the user can choose a position within the palpation ring 124 to access the reservoir 104 using an access needle.

Penetrating the septum 120 with an access needle, within the top-most edge 92 of the septum 120 can ensure that the access needle can access the reservoir 104. Worded differently, a horizontal surface area of the inner surface 128 of the septum 120 can be equal to or slightly smaller than the horizontal surface area of the reservoir 104. As such, penetrating the septum at any point within the inner surface 128, defined by the top-most edge 92 can ensure the user can access the reservoir 104. In an embodiment, a radially outer-most edge 94 of the palpation ring 124 can extend beyond a radially outer-most edge of the reservoir 104. Worded differently, a horizontal surface area defined by the palpation ring 124 can be equal to or slightly larger than the horizontal surface area of the reservoir 104. In an embodiment, the outer surface 126 of the palpation ring 124 can define a smooth transition surface between the outer surface of the port body 102 and the palpation ring 124 and can mitigate stretching or skin erosion.

In an embodiment, as shown in FIG. 1B a cross-sectional shape of the inner surface 128 of the palpation ring 124 can define a relatively steeper incline proximate the top-most edge 92 before tapering off to a relatively shallower incline towards the central transverse axis 90. As such, the inner surface 128 of the palpation ring 124 can define a relatively wide bowl shape and can provide a relatively larger horizontal surface area over which the user can penetrate the septum 120 to access the reservoir 104. Advantageously, the relatively wide inner surface 128 of the palpation ring 124 can allow the user to access the reservoir at different positions mitigating the formation of scar tissue, or degradation of the skin surface tissues disposed thereover, from repeated access. As used herein, an “incline” of a surface is defined in relation to a horizontal plane.

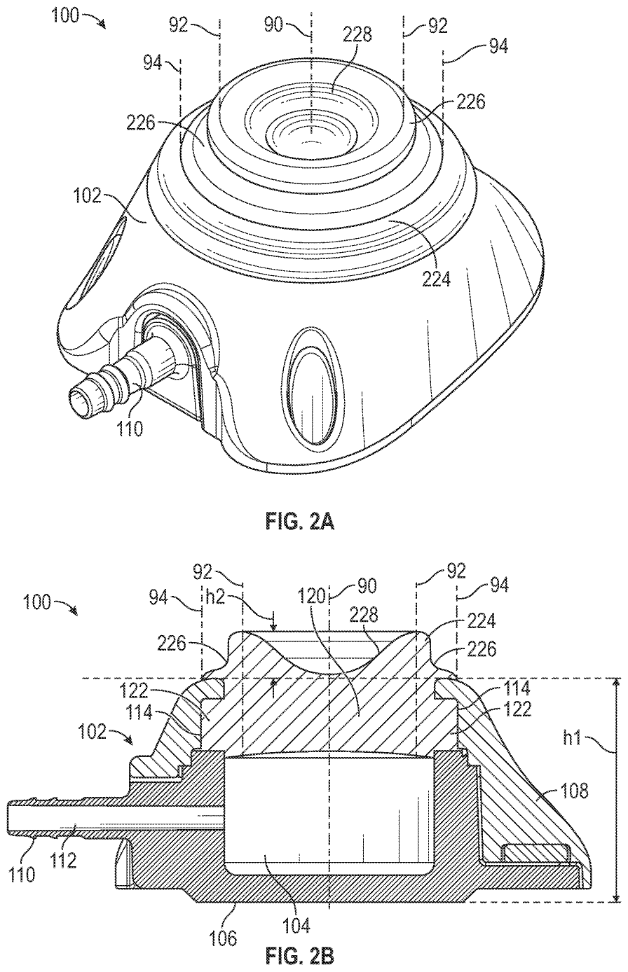

FIGS. 2A-2B show an embodiment of the port 100 including a septum 120 having a concave top surface and a palpation ring 224. The palpation ring 224 can define an outer surface 226 extending between an upper-most edge 92 and a radially outer-most edge 94, as well as an inner surface 228 extending between the upper-most edge 92 and a central transverse axis 90, as described herein.

In an embodiment, a cross-sectional shape of the inner surface 228 of the palpation ring 220 can define relatively more consistent incline extending between the top-most edge 92 and the central transverse axis 90. Advantageously, the relatively consistent incline of the inner surface 228 can direct a user towards the central transverse axis 90 and facilitate accuracy when accessing the reservoir 104. This can be of particular importance when accessing reduced size port systems, e.g. port systems that are disposed in a limb such as an arm or the like.

In an embodiment, the outer surface 226 of the palpation ring 224, extending from the topmost edge 92 of the palpation ring 224 radially outwards to a radially outer-most edge 94, can define a concave cross-sectional shape. In an embodiment, a section of the outer surface 226 disposed adjacent the top-most edge 92 can define a relatively steep incline before tapering out to a relatively shallow incline adjacent the radially outer-most edge 94. In an embodiment, a section of the outer surface 226 disposed adjacent the top-most edge 92 can define a substantially vertical incline before tapering out to a relatively shallow incline adjacent the radially outer-most edge 94. Advantageously, the concave outer surface 226 of the palpation ring 224 can reduce a contact surface area between the palpation ring outer surface 226 and the skin surface tissues extending over the port 100, thereby mitigating contact therebetween and mitigating skin erosion. Advantageously, the concave outer surface 226 of the palpation ring 224 including the steep or vertical section can provide improved definition to the palpation ring 224 providing a more easily identified palpation feature when placed subcutaneously.

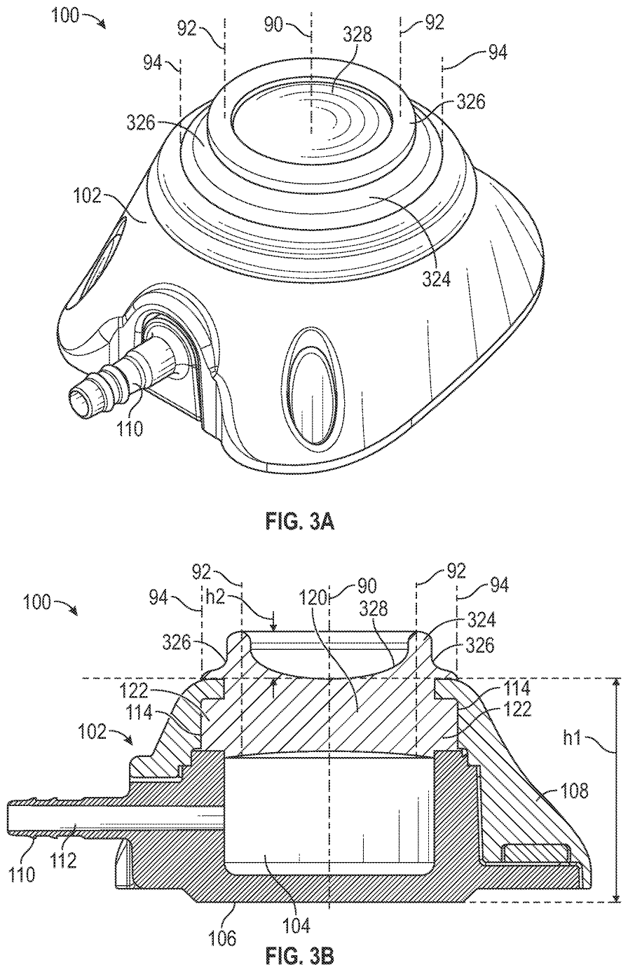

FIGS. 3A-3B show an embodiment of the port 100 including a septum 120 having a concave top surface and a palpation ring 324. The palpation ring 324 can define an outer surface 326 extending between an upper-most edge 92 and a radially outer-most edge 94, as well as an inner surface 328 extending between the upper-most edge 92 and a central transverse axis 90, as described herein. In an embodiment, the outer surface 326 of the palpation ring 324 can define a concave cross-sectional shape. In an embodiment, a section of the outer surface 326 disposed adjacent the top-most edge 92 can define a relatively steep, or vertical, incline before tapering out to a relatively shallow incline adjacent the radially outer-most edge 94.

In an embodiment, the inner surface 328 of the palpation ring 324 can also define a relatively steep, or vertical, section adjacent the top-most edge 92, before tapering off to a relatively shallow incline adjacent the central vertical axis 90. Advantageously, the vertical sections of the inner surface 328 and the outer surface 326, disposed either side of the top-most edge 92, can define a pronounced palpation ring 324 to facilitate palpation of the port 100 when placed subcutaneously. Further the concave inner surface 328 and the concave outer surface 326 can provide a reduced contact surface area between the port 100 and skin tissues disposed thereover to mitigate skin erosion.

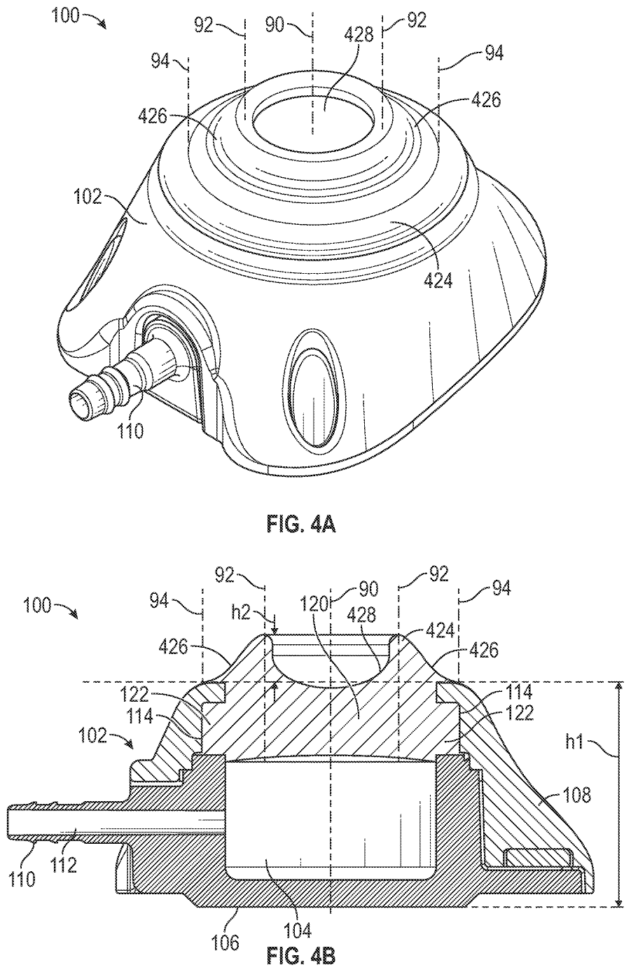

FIGS. 4A-4B show an embodiment of the port 100 including a septum 120 having a concave top surface and a palpation ring 424. The palpation ring 424 can define an outer surface 426 extending between an upper-most edge 92 and a radially outer-most edge 94, as well as an inner surface 428 extending between the upper-most edge 92 and a central transverse axis 90, as described herein.

In an embodiment, the outer surface 426 of the palpation ring 424 can extend at a substantially continuous incline between the topmost edge 92 and the radially outer-most edge 94, to define frusto-conical shape to the palpation ring 124. Advantageously, the outer surface 426 can provide a smooth transition between the port body 102 and the top-most edge 92 of the palpation ring 424 to mitigate pressure points on skin surface tissues stretched thereover and mitigate skin erosion.

In an embodiment, the inner surface 428 of the palpation ring 424 can define a concave cross-sectional profile and can include a vertical section disposed adjacent the top-most edge 92. The inner surface 428 can then provide a continuous change in incline between the vertical section and the central vertical axis 90 to provide a rounded concave section to the inner surface 416 of the palpation ring 424. Advantageously, the inner surface 428 can both direct a user towards the central transverse axis 90 and provide a relatively larger horizontal surface area to access the reservoir 104 at different positions, mitigating the build-up of scar tissue from repeated needle sticks at the same position.

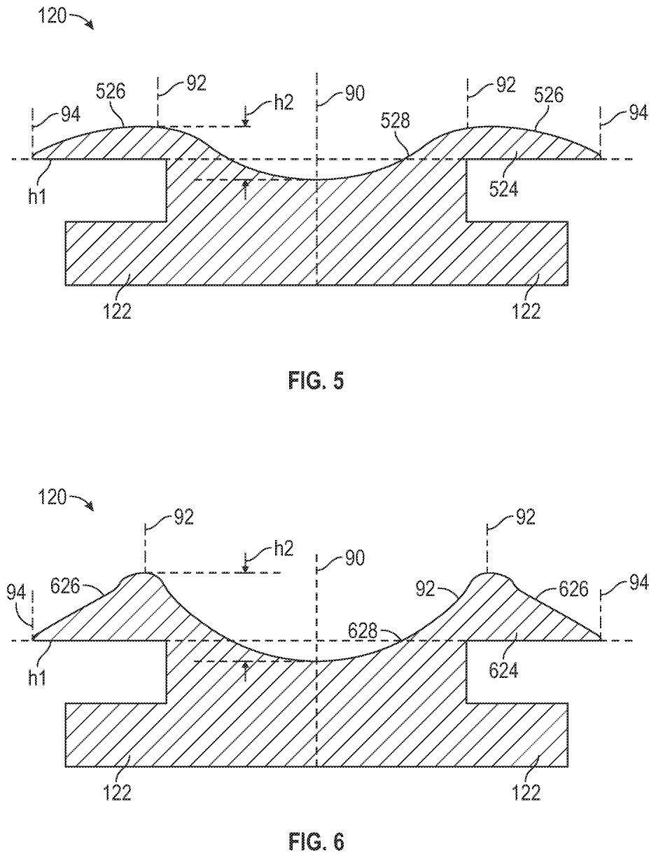

FIGS. 5-8 show cross-sectional views of embodiments of the septum 120 for use with a low profile port 100, and including a concave section to the top surface and a palpation ring. In general, the septum 120 can define a radially symmetrical shape and include a concave section to the upper surface extending between a top-most edge 92 and a central transvers axis 90. As such, the top-most edge 92 can define a palpation ring extending continuously about the central transverse axis 90, as described herein. In an embodiment, the palpation ring can include an inner surface extending between the top-most edge 92 and the central transverse axis 90, and an outer surface extending between the top-most edge 92 and the radially outer-most edge 94, as described herein. In an embodiment, the top-most edge 92 of the palpation ring can extend transversely outwards from the top most edge of the port body 102 to define the top-most edge of the port 100 as a whole.

In an embodiment, a portion of the inner surface can extend transversely below a top-most edge of the port body 102. As such, the top surface of the septum 120 can define a concave surface extending over a transverse height (h2) while reducing the overall transverse height of the port 100, i.e. the height of the port 100=<height (h1)+height (h2). Advantageously, the overall transverse height of the port 100 can be reduced without reducing the effectiveness (depth) of the palpation ring and concave section to palpation.

FIG. 5 shows an embodiment of a septum 120 including a concave section and a palpation ring 524. The outer surface 526 of the palpation ring 524 can define a convex cross-sectional profile. The inner surface 528 of the palpation ring 524 can define a sigmoid curved cross-sectional profile, curving in a first direction adjacent the top-most edge 92 before transitioning through an inflection point and curving in an second direction, opposite the first direction, adjacent the central transverse axis 90.

FIG. 6. shows an embodiment of a septum 120 including a concave section and a palpation ring 624. The outer surface 626 of the palpation ring 624 can define a consistent incline between the top-most edge 92 and the outer-most edge 94. The inner surface 628 of the palpation ring 624 can define a consistent change in incline between a relatively steep, or vertical, incline adjacent the top-most edge 92 and a relatively shallow incline adjacent the central transverse axis 90.

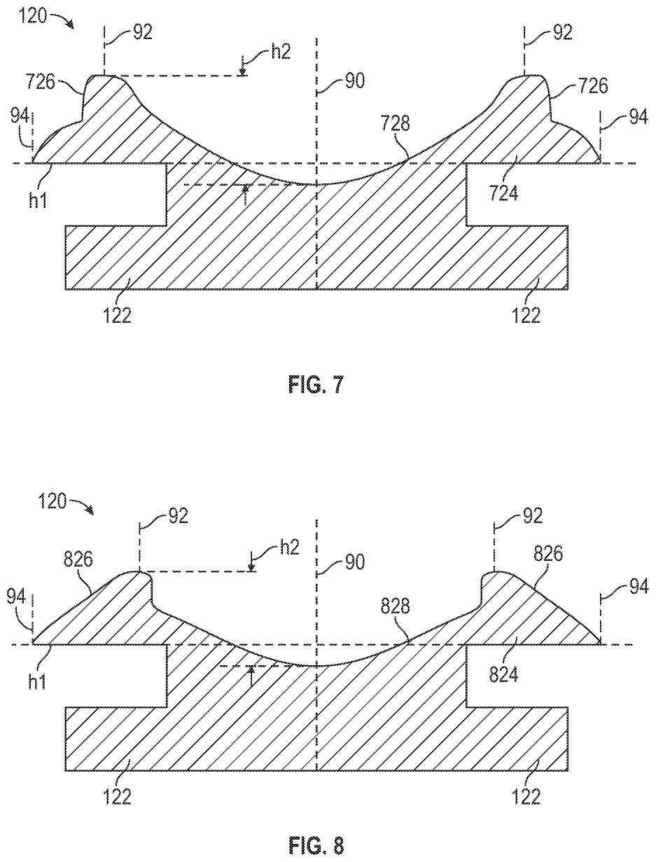

FIG. 7. shows an embodiment of a septum 120 including a concave section and a palpation ring 724. The outer surface 726 of the palpation ring 724 can include a substantially vertical section adjacent the top-most edge 92 and a convex section adjacent the outer-most edge 94. The inner surface 728 of the palpation ring 724 can include a consistent change in incline between a relatively steep incline adjacent the top-most edge 92 and a relatively shallow incline adjacent the central transverse axis 90.

FIG. 8. shows an embodiment of a septum 120 including a concave section and a palpation ring 824. The outer surface 826 of the palpation ring 824 can include a consistent incline between the top-most edge 92 and the outer-most edge 94. The inner surface 828 of the palpation ring 824 can include a substantially vertical section adjacent the top-most edge 92 and a section including a consistent change in incline between a relatively steep incline adjacent the top-most edge 92 and a relatively shallow incline adjacent the central transverse axis 90.

While some particular embodiments have been disclosed herein, and while the particular embodiments have been disclosed in some detail, it is not the intention for the particular embodiments to limit the scope of the concepts provided herein. Additional adaptations and/or modifications can appear to those of ordinary skill in the art, and, in broader aspects, these adaptations and/or modifications are encompassed as well. Accordingly, departures may be made from the particular embodiments disclosed herein without departing from the scope of the concepts provided herein.

1. A vascular access port, comprising:
a port body having a reservoir; and
a septum positioned over the reservoir and captured by the port body, the septum comprising:
a concave top surface configured for penetration by a needle to permit fluid communication between the needle and the reservoir; and
a continuous rim extending around a central transverse axis of the concave surface.

2. The vascular access port according to claim 1, wherein the septum defines a radially symmetrical shape extending about the central transverse axis.

3. The vascular access port according to claim 1, wherein the continuous rim extends transversely outwards from an upper-most surface of the port body.

4. The vascular access port according to claim 1, wherein the upper surface of the continuous rim defines an upper-most surface of the vascular access port.

5. The vascular access port according to claim 1, wherein the septum including the continuous rim is formed of a compliant material selected from a group consisting of a plastic, polymer, elastomer, organic or synthetic rubber, or silicone rubber.

6. The vascular access port according to claim 1, wherein the continuous rim includes an outer surface, the outer surface extends between an upper-most edge and a radially outer-most edge, and an inner surface, the inner surface extends between the upper-most edge and the central transverse axis.

7. The vascular access port according to claim 6, wherein the outer surface defines a concave cross-sectional profile.

8. The vascular access port according to claim 6, wherein the outer surface defines a convex cross-sectional profile.

9. The vascular access port according to claim 6, wherein the outer surface defines a continuous incline relative to the central transverse axis.

10. The vascular access port according to claim 6, wherein the inner surface defines a convex cross-sectional profile.

11. The vascular access port according to claim 6, wherein the inner surface includes sigmoid cross-sectional profile including a convex section and a concave section.

12. The vascular access port according to claim 6, wherein the inner surface includes one of a discontinuous change in incline or a continuous change in incline.

13. The vascular access port according to claim 6, wherein one or both of the outer surface and the inner surface includes a vertical section extending parallel to the central transverse axis.

14. The vascular access port according to claim 1, wherein the concave surface includes a portion that extends below an upper-most surface of the port body.

15. The vascular access port according to claim 1, wherein the septum includes flange extending radially outward from a side surface of the septum.

16. The vascular access port according to claim 1, wherein the port is configured to be disposed subcutaneously and further includes a catheter coupled to a port stem and in fluid communication with the reservoir, the distal tip of the catheter disposed within a vasculature.

17. A method of manufacturing a vascular access port, comprising:
forming a port body defining a reservoir; and
coupling a septum with the port body, the septum positioned over the reservoir and comprising:
a top surface having a concave portion and configured for penetration by a needle to permit fluid communication between the needle and the reservoir; and
a continuous rim extending around a central transverse axis of the concave surface.

18. The method according to claim 17, wherein the septum defines a radially symmetrical shape extending about the central transverse axis.

19. The method according to claim 17, wherein the continuous rim extends transversely outwards from an upper-most surface of the port body.

20. The method according to claim 17, wherein the upper surface of the continuous rim defines an upper-most surface of the vascular access port.

21. The method according to claim 17, wherein the septum including the continuous rim is formed of a compliant material selected from a group consisting of a plastic, polymer, elastomer, organic or synthetic rubber, or silicone rubber.

22. The method according to claim 17, wherein the continuous rim includes an outer surface extending between an upper-most edge and a radially outer-most edge, and an inner surface extend between the upper-most edge and the central transverse axis.

23. The method according to claim 22, wherein the outer surface defines a concave cross-sectional profile.

24. The method according to claim 22, wherein the outer surface defines a convex cross-sectional profile.

25. The method according to claim 22, wherein the outer surface defines a continuous incline.

26. The method according to claim 22, wherein the inner surface defines a convex cross-sectional profile.

27. The method according to claim 22, wherein the inner surface includes sigmoid cross-sectional profile including a convex section and a concave section.

28. The method according to claim 22, wherein the inner surface includes one of a discontinuous change in incline or a continuous change in incline.

29. The method according to claim 22, wherein one or both of the outer surface and the inner surface includes a vertical section extending parallel to the central transverse axis.

30. The method according to claim 17, wherein the concave surface includes a portion that extends below an upper-most surface of the port body.

31. The method according to claim 17, wherein the septum includes flange extending radially outward from a side surface of the septum.

32-35. (canceled)
